(19) United States
(12) Patent Application Publication
(10) Pub. No.: US 2015/0328106 A1
(43) Pub. Date: Nov. 19, 2015

(54) ANTIMICROBIAL HAND WASH COMPOSITION

(71) Applicant: The Dial Corporation, Scottsdale, AZ (US)

(72) Inventors: Nancy D. Rodgers, Chandler, AZ (US); Janice L. Fuls, Fountain Hills, AZ (US); Jessica Killian, Scottsdale, AZ (US); Kanani Guerra, Phoenix, AZ (US)

(73) Assignee: The Dial Corporation, Scottsdale, AZ (US)

(21) Appl. No.: 14/279,417

(22) Filed: May 16, 2014

(51) Int. Cl. A61K 8/41; A61K 8/30; A61K 8/27; A61Q 19/10

(52) U.S. Cl. CPC A61K 8/41; A61Q 19/10; A61K 8/30; A61K 8/27

(57) ABSTRACT

Methods and apparatus are provided for an antimicrobial hand wash composition. The antimicrobial hand wash composition includes a cationic antimicrobial active ingredient. The antimicrobial hand wash composition further includes a number of surfactants including at least two surfactants compatible with cationics with at least one of the surfactants including an alkamine oxide. Further, the antimicrobial hand wash composition includes a hydrocolloid. The antimicrobial hand wash composition further includes a zinc salt including at least one of zinc sulfate and zinc gluconate.

### FIELD OF THE INVENTION

The present invention generally relates to an antimicrobial hand wash composition, and more particularly relates to an antimicrobial hand wash composition that exhibits a 3 log 10 reduction in bacteria on a skin surface after ten hand washes.

### BACKGROUND OF THE INVENTION

Antimicrobial hand wash compositions are used to help reduce the risk of disease by decreasing bacteria on the skin in several settings. Such setting may include healthcare settings, food handling settings, and settings in a home. To determine the antibacterial efficacy of an antimicrobial hand wash composition, a health care personnel hand wash method may be performed. The health care personnel hand wash method is performed to determine the effectiveness of the antimicrobial hand wash composition for reducing bacteria, such as transient microbial flora, on a skin surface initially and then over consecutive washes.

Accordingly, it is desirable to have an antimicrobial hand wash composition which reduces bacteria on a skin surface. In addition, it is desirable to have an antimicrobial hand wash composition that reduces bacteria on a skin surface by 3 log 10 after ten washes as measured by the health care personnel hand wash method. Furthermore, other desirable features and characteristics of the present invention will become apparent from the subsequent detailed description of the invention and the appended claims, taken in conjunction with the background of the invention.

### BRIEF SUMMARY OF THE INVENTION

An antimicrobial hand wash composition includes a cationic antimicrobial active ingredient, a number of surfactants including at least two surfactants compatible with cationics with at least one of the surfactants including an alkamine oxide, a hydrocolloid, and a zinc salt including at least one of zinc sulfate and zinc gluconate.

An antimicrobial hand wash product includes a container, and an antimicrobial hand wash composition disposed within the container. The antimicrobial hand wash composition includes a cationic antimicrobial active ingredient, a number of surfactants including at least two surfactants compatible with cationics with at least one of the surfactants including an alkamine oxide, a hydrocolloid, a moisturizer, a zinc salt including at least one of zinc sulfate and zinc gluconate, and an aqueous medium in which the cationic antimicrobial active ingredient, the number of surfactants, the hydrocolloid, the moisturizer, and the zinc salt are distributed.

An antimicrobial hand wash composition includes a cationic antimicrobial active ingredient forming between 0.10 and 0.30 weight percent of the antimicrobial hand wash composition, a number of surfactants including at least two surfactants compatible with cationics with at least one of the surfactants including an alkamine oxide forming between 1.00 and 2.00 weight percent of the antimicrobial hand wash composition, a hydrocolloid forming between 0.01 and 1.00 weight percent of the antimicrobial hand wash composition, a moisturizer forming between 1.00 and 3.00 weight percent of the antimicrobial hand wash composition, and a zinc salt forming between 0.01 and 2.00 weight percent of the antimicrobial hand wash composition. The zinc salt has a log D value less than 0 at a pH of 5.5. The zinc salt has a log P value less than 0. The antimicrobial hand wash composition has a molar ratio of zinc salt to a ligand that is between 3:20 and 7:20.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Antimicrobial hand wash compositions are used to decrease the risk of disease by reducing bacteria on the skin in many settings. Such setting may include healthcare setting, food handling setting, and setting in a home. However, some antimicrobial hand wash compositions are more effective in reducing the bacteria on a skin surface than other antimicrobial hand wash compositions. As a result, an antimicrobial hand wash composition that reduces more bacteria on a skin surface verses activity demonstrated in in-vitro type studies would be more desirable to use in several of the settings.

Accordingly, the principles described herein provide a composition that exhibits a 3 log 10 reduction in bacteria on a skin surface after ten washes using a health care personnel hand wash method. More specifically, the present specification describes an antimicrobial hand wash composition that contains zinc salts to achieve the 3 log 10 reduction in bacteria on a skin surface after ten washes as measured by the health care personnel hand wash method. The zinc salt may include zinc sulfate and zinc gluconate. The zinc salt includes characteristics such as a log D value less than 0 at a pH of 5.5, a log P value less than 0, and a molar ratio of zinc salt to a ligand that is between 3:20 and 7:20. In other words, the characteristics of the zinc salt allows the antimicrobial hand wash composition to exhibit a 3 log 10 reduction in bacteria on a skin surface after ten washes using the health care personnel hand wash method.

The health care personnel hand wash method is used to determine the effectiveness of the antimicrobial hand wash composition for reducing bacteria, such as transient microbial flora. In one example, the health care personnel hand wash method includes a number of procedures. As will be described below, the procedures may include a hand cleansing procedure, a hand contamination procedure, a wash and rinse procedure, a bacterial recovery procedure, an enumeration of bacteria in sampling solution procedure, a determination of reduction procedure, and a comparison of test material procedure.

As mentioned above, the health care personnel hand wash method includes a hand cleansing procedure. After subjects have refrained from using antimicrobial formulations for at least 7 days, they perform a 30 second cleansing wash. This procedure removes oil and dirt and familiarizes panelists with the washing technique. For this and all other washes and rinses, the water temperature is adjusted to 40±2 degrees Celsius (C) and the water flow rate to 4 liters (L) per minute. In one example, the flow rate may be determined via a number of flow gauges.

As mentioned above, the health care personnel hand wash method includes a hand contamination procedure. The subject's hands are contaminated with a test organism such as bacteria. A liquid suspension of the test organism containing between 5.0×10̂8 and 1.0×10̂9 colony forming unit (cfu) per mL is used. A 1.5 mL aliquot of the test organism suspension is dispensed into the subjects' cupped hands. This aliquot is rubbed over the entire surface of the hands for 20±5 seconds not reaching above the wrist. The hands are then held motionless away from the body and allowed to air dry for approximately 30±5 seconds. To continue the contamination of the hands, an additional 1.5 mL aliquot of the test organism suspension is dispensed into the hands, distributed over the hands for 20±5 seconds, and air dried for 30±5 seconds. To complete the contamination, a final 1.5 mL aliquot of test organism suspension is dispensed into the hands, distributed over the hands for 20±5 seconds, and air dried for 90±5 seconds. The total test organism suspension applied to the hands is 4.5 mL. Contamination may take approximately 5 minutes. This method of contamination minimizes the loss of test organism while spreading.

Further, the subjects' hands are contaminated with the test organism prior to a baseline bacterial sample collection and prior to each washing with the test material. The number of repeated test washes may be reduced or eliminated at the discretion of the investigator. A baseline sample is taken after contamination to determine the number of marker organisms surviving on the hands. Bacterial sampling will follow the procedures outlined later on in this specification.

As mentioned above, the health care personnel hand wash method includes a wash and rinse procedure. In one example, a wash and rinse procedure is performed for liquid formulations. The wash and rinse procedure may dispense 5 mL of test material into cupped hands within 10 seconds of completing the drying step. This is spread over hands and lower third of forearms. The subject sparingly wets the contaminated hands by rapidly passing them one time through the tap water. This process should be performed in less than 1 second. The subject further washes, in a vigorous manner for 30±5 seconds, all surfaces of the hands and the lower third of the forearm. Caution should be exercised to retain the test material in the hands. If the lather becomes too dry, a small amount of water may be added to maintain lather. Rinse thoroughly from fingertips to elbows under 40±2 degrees C. tap water for 30±5 seconds. Caution should be exercised to avoid contact with the sink and fixtures to eliminate recontamination from the sink surfaces and to avoid rubbing hands and forearms during the rinsing process. The subject's hands and forearms are lightly patted dry with paper toweling.

For waterless formulations, the wash and rinse procedure may be as follows. Dispense 3 mL of test material into cupped hands within 10 seconds of completing the drying step. Within 10 seconds, distribute test material over all surfaces of the hands and the lower third of the forearms. Continue rubbing in a vigorous manner for 30±5 seconds. Caution should be exercised to retain the test material in the hands The subject's hands may be held upright and motionless prior to the bacterial recovery procedure.

For solid formulations, the wash and rinse procedure may be as follows. Sparingly wet contaminated hands and forearms with 40±2 degrees C. tap water. Wet the product. Rub the product between the hands and on the forearms for 15±3 seconds. Place product aside. Lather lower third of forearms and hands for an additional 30±5 seconds. If the lather becomes too dry, a small amount of water may be added to maintain lather. Rinse thoroughly from fingertips to elbows under 40±2 degrees C. tap water for 30±5 seconds. Caution should be exercised to avoid contact with the sink and fixtures to eliminate contamination from the sink surfaces. The subject's hands and forearms are lightly patted dry with paper toweling.

As mentioned above, the health care personnel hand wash method may include a bacterial recovery procedure. The bacterial recovery procedure may be as follows. Within one minute after specified washes, place gloves used for sampling on the hands of the subject. Add 75 mL of sampling solution with neutralizer to each glove and secure gloves above the wrist. Within one minute of donning gloves, uniformly massage all surfaces of the hand for one minute ±5 seconds, paying particular attention to the fingers and flipping the hand after 30 seconds to ensure both the palm and back of the hand are thoroughly massaged. Within one minute of completing the massage, aseptically retrieve a 3 to 5 mL sample of the fluid in the glove by pulling the glove away from the wrist, inserting a pipet into the finger region of the glove, and withdrawing the fluid. The first dilution is to be made in dilution fluid with appropriate neutralizer within 10 seconds of removing the 3 to 5 mL from the glove. The plating of the recovered sampling solution is completed within 30 minutes after sampling.

As mentioned above, the health care personnel hand wash method may include an enumeration of bacteria in the sampling solution procedure. The enumeration of bacteria in the sampling solution procedure may be as follows. Enumerate a Serratia marcescens (S. marcescens) in the recovered sampling solution using standard microbiological techniques, such as membrane filtration or spread plating. The pour plate technique is not recommended because subsurface S. marcescens colony forming units may not exhibit the red pigment. Prepare dilutions of the recovered sampling solution in dilution fluid. Use soybean-casein digest agar with suitable inactivator as recovery medium. Incubate prepared plates 48±4 hours at 25±2 degrees C. Standard plate counting procedures are used to count only the red pigmented S. marcescens. Enumerate Escherichia coli (E. coli) in the sampling solution using standard microbiological techniques, such as membrane filtration, pour or spread plating. Prepare dilutions of the recovered sampling solution in dilution fluid. Use soybeancasein digest agar with suitable inactivator and indicator (MUG10) as a recovery medium. Incubate prepared plates 18 to 24 hours at 35±2 degrees C. Standard plate counting procedures are used to count only the E. coli colonies.

As mentioned above, the health care personnel hand wash method may include a determination of reduction procedure. The determination of reduction procedure may be as follows. Convert plate counts, cfu per hand, to log 10. Average left and right hands for each sampling interval. Determine log 10 reductions at each recovery interval or wash using the following formula: Log 10 Reduction at Sampling Interval=Log 10 Baseline Recovery—Log 10 Sampling Interval.

As will be describe below, the health care personnel hand wash method may be used as a standard to determine the effectiveness of the antimicrobial hand wash composition for reducing bacteria, such as transient microbial flora on a skin surface. Further, in this specification, the reduction in bacteria on the skin surface is determined after a first hand was washed using the health care personnel hand wash method and after ten washes using the health care personnel hand wash method.

The antimicrobial hand wash composition includes a cationic antimicrobial active ingredient. As used in the present specification and in the appended claims, a cationic antimicrobial active ingredient refers to any compound used to prevent, reduce, eliminate, or otherwise combat bacteria on a skin surface such as the hands. For example, a cationic antimicrobial active ingredient may prevent bacteria from forming on the skin surface. A cationic antimicrobial active ingredient may also reduce bacteria currently present on the skin surface. In some examples, the cationic antimicrobial active ingredient forms from 0.10 to 0.30 weight percent of the antimicrobial hand wash composition. For example, the cationic antimicrobial active ingredient may form 0.30 weight percent of the antimicrobial hand wash composition.

The antimicrobial hand wash composition includes a number of surfactants including at least two surfactants compatible with cationics with at least one of the surfactants including an alkamine oxide. In one example, cationics refers to an ion or a group of ions having a positive charge. A surfactant has a hydrophobic end and a hydrophilic end. The hydrophobic end allows the surfactant to interact with uncharged molecules, such as oils. The hydrophilic end facilitates the interaction of the molecule with charged or polar molecules, such as water. The hydrophilic end is used to classify surfactants, which may be cationic, non-ionic, or amphoteric. In one example, a surfactant that is compatible with cationics may be cationic surfactants. Cationic surfactants are those that have a positively charged hydrophilic end, such as a quaternary amine. In another example, a surfactant that is compatible with cationics may be non-ionic surfactants. Non-ionic surfactants have a hydrophilic end which may be charge neutral, such as an ethoxylate, glycoside, or poly-ol; such surfactants may not be sensitive to water hardness. In yet another example, a surfactant that is compatible with cationics may be amphoteric surfactants. Amphoteric or zwitterionic surfactants may have both a positive and negative charge on their hydrophilic ends, such as amine oxides, sultaines, or betaines. In some examples, the surfactants include alkamine oxides.

In accordance with various examples, an alkamine oxide useful in the antimicrobial hand wash composition contains at least one long hydrocarbon chain containing at least eight carbon atoms. One class of amine oxides is the alkyl di (lower alkyl) amine oxides, wherein the alkyl group contains 8 to 22, and preferably about 10 to about 16, carbon atoms, and can be straight or branched chain, saturated or unsaturated. The lower alkyl groups contain 1 to 7 carbon atoms, and typically are methyl. Specific examples include, but are not limited to, lauryl dimethyl amine oxide, myristyl dimethyl amine oxide, dimethyl cocoamine oxide, dimethyl (hydrogenated tallow) amine oxide, myristyl/palmityl dimethyl amine oxide, myristyl/lauryl dimethyl amine oxide, cetyl dimethyl amine oxide, stearyl dimethyl amine oxide, and myristyl/cetyl dimethyl amine oxide.

Another class of useful amine oxides includes alkyl di (hydroxy lower alkyl) amine oxides in which the alkyl group contains 8 to 22, and preferably about 10 to about 16 carbon atoms, and can be straight or branched chain, saturated or unsaturated. Specific examples, include, but are not limited to, bis(2-hydroxyethyl) cocoamine oxide, bis(2-hydroxyethyl) tallow amine oxide, and bis(2-hydroxyethyl) stearylamine oxide.

Additional useful amine oxides are termed alkamidopropyl di (lower alkyl) amine oxides in which the alkyl group contains 8 to 22, and preferably about 10 to about 16 carbon atoms, and can be straight or branched chain, saturated or unsaturated. Examples are cocoamidopropyl dimethyl amine oxide and tallowamidopropyl dimethyl amine oxide. Further useful amine oxides are termed alkylmorpholine oxides in which the alkyl group contains 8 to 22, and preferably about 10 to about 16, carbon atoms, and can be straight or branched chain, saturated or unsaturated.

The above classes of alkamine oxide surfactants contain a C8-C22 alkyl group selected from, for example, octyl, decyl, undecyl, lauryl, tridecyl, myristyl, cetyl, stearyl, isostearyl, olcyl, and mixtures thereof. Examples of amine oxide surfactants include, but are not limited to, decyl dimethylamine oxide, lauryl dimethylamine oxide, stearyl dimethylamine oxide, oleyl dimethylamine oxide, coco dihydroxyethylamine oxide, cetyl N,N-dihydroxyethylamine oxide, oleyl N,N-dihydroxyethylamine oxide, cocamine oxide, cocamidopropylamine oxide, lauramidopropylamine oxide, oleamine oxide, oleamidopropylamine oxide, wheat germaraidopropylamine oxide, isostearamido-propylamine oxide, stearamine oxide, stearamido-propylamine oxide, cocomorpholine oxide, decylamine oxide, dihydroxyethyl C s-C ₁₀ alkoxypropylamine oxide, dihydroxyethyl Cg-Ci i alkoxypropylamine oxide, dihydroxyethyl C ₁2-Cn alkoxypropylamine oxide, dihydroxyethyl cocamine oxide, dihydroxyethyl stearamine oxide, dihydroxyethyl tallowamine oxide, hydrogenated tallow amine oxide, hydroxyethyl hydroxypropyl C 12-C 15 alkoxypropylamine oxide, isostearamidopropyl morpholine oxide, myristamidopropylamine oxide, myristamine oxide, pahnitamidopropylamine oxide, palmitamine oxide, PEG-3 lauramine oxide, tallow amidopropylamine oxide, tallow amine oxide, undecylenamidopropylamine oxide, and mixtures thereof. Preferred alkamine oxide surfactants are the alkyl di (lower alkyl) amine oxides in which the alkyl group contains about 12 to about 16 carbon atoms, including lauramine oxide, myristamine oxide, cocamine oxide, cetamine oxide, and mixtures thereof.

In accordance with other examples of the present specification, the composition contains a blend of alkamine oxide surfactants. In some examples, a first component of the alkamine oxide blend contains twelve or fewer carbon atoms and a second component contains more than twelve carbon atoms.

Examples of amphoteric surfactants that can be included in the composition include, but are not limited to, betaines, hydroxypropylsultaines, amine oxides, n-alkylaminopropionates, n-alkylimino dipropionates, phosphobetaines, phosphitaines, imidazolines, and mixtures thereof. Examples of specific amphoteric surfactants include, but are not limited to, cocamidopropyl betaine, lauramidopropyl betaine, coco/oleamidopropyl betaine, coco betaine, oleyl betaine, cocamidopropyl hydroxysultaine, tallowamidopropyl hydroxysultaine, dihydroxyethyl tallow glycinate, cocodimethylcarboxymethylbetaine, lauryldimethylcarboxymethylbetaine, lauryldimethylcarboxyethylbetaine, cetyldimethylcarboxymethylbetaine, lauryl-bis-(2-hydroxyethyl)carboxymethylbetaine, oleyldimethylgamma-carboxypropylbetaine, lauryl-bis-(2-hydroxypropyl)-carboxyethylbetaine, cocodimethylpropylsultaine, stearyldimethylpropylsultaine, laurylbis-(2-hydroxyethyl)propylsultaine, cocoamidodimethylpropylsultaine, stearylamidodimethylpropylsultaine, laurylamido-bis-(2-hydroxyethyl) propylsultaine, cocamido disodium 3-hydroxypropyl phosphobetaine, lauric myristic amido disodium 3-hydroxypropyl phosphobetaine, lauric myristic amido glyceryl phosphobetaine, lauric myristic amido carboxy disodium 3-hydroxypropyl phosphobetaine, cocoamido propyl monosodium phosphitaine, lauric myristic amido propyl monosodium phosphitaine, and mixtures thereof. Particularly useful amphoteric surfactants are cocamidopropyl betaine. In general, however, any amphoteric surfactant can be included in the composition of the antimicrobial hand wash composition as long as the stability, the conditioning, and the antimicrobial efficiency of the composition are not adversely affected.

While specific reference has been made to certain surfactants, the number of surfactants in the antimicrobial hand wash composition may include any type, combination or mixture of surfactants. The surfactant may form between 1.00 to 4.00 weight percent of the antimicrobial hand wash composition.

The antimicrobial hand wash composition includes a hydrocolloid. In one example, the hydrocolloid may be methylcellulose. In other example, the hydrocolloid may be other substances. Regardless of the type of hydrocolloid used in the antimicrobial hand wash composition, the hydrocolloid is used to form a gel with an aqueous medium of the antimicrobial hand was composition. In some examples, the hydrocolloid forms between 0.01 and 1.00 weight percent of the antimicrobial hand wash composition.

The antimicrobial hand wash composition includes a zinc salt. In one example, the zinc salt may include at least one of zinc sulfate and zinc gluconate. As will be described in this specification, for the antimicrobial hand wash composition to exhibit a 3 log 10 reduction in bacteria on a skin surface, the zinc salt includes a number of defining characteristics. In one example, a 1 log 10 refers to a ten times reduction in the number of bacteria. A 2 log 10 refers to a one-hundred times reduction in the number of bacteria. Further, a 3 log 10 refers to a one-thousand times reduction in the number of bacteria

Compounds may have ionizable groups that exist in solution as a mixture of different ionic forms. The ionization of those groups, thus the ratio of the ionic forms, depends on the pH. The octanol-water distribution coefficient, log D, represents the compounds at any pH value. In one example, the zinc salt has a log D value less than 0 at a pH of 5.5. Further, the log D may be represented by equation 1 as seen below.

$$log {D}_{oct \text{/} wat} = log ( \frac{{[ solute ]}_{octanol}}{{[ solute ]}_{water}^{ionized} + {[ solute ]}_{water}^{neutral}} ) . \qquad ( Equation 1 )$$

The characteristics of the zinc salt may be further defined as a log P value. A log P value, identified as partition coefficient, is a ratio of concentrations of an un-ionized compound in two phases of immiscible solvents, such as water and n-octanol, at equilibrium. Further, the log P value is the 10-base logarithmic measure of the coefficient. In one example, the zinc salt has a log P value less than 0. The log P may be represented by equation 2 as seen below.

$$log {D}_{oct \text{/} wat} = log ( \frac{{[ solute ]}_{octanol}}{{[ solute ]}_{water}^{un \text{-} ionized}} ) . \qquad ( Equation 2 )$$

The characteristics of the zinc salt may be further defined by a molar ratio of zinc salt to a ligand. In one example, a ligand is an ion or molecule binding to form a complex. In this specification, the ligands are the molecules that the zinc is complexing with. For example, the ligands may be complexing with zinc chloride, zinc gluconate, or zinc sulfate. In some example, a molar ratio of zinc salt to a ligand is between 3:20 and 7:20.

In some example, different zinc salts may contribute different amounts of zinc ions to the antimicrobial hand wash composition. For example, 0.05 percent of zinc salt may be added to a 100 gram sample size of the antimicrobial hand wash composition. Once the zinc salt if fully dissolved, the amount of the zinc ions in 100 grams of the antimicrobial hand wash composition at a level of 0.05 percent is 1.14e−4 grams for the zinc sulfate. Further, an amount of the zinc ions in 100 grams of the antimicrobial hand wash composition at a level of 0.05 percent is 7.17e−5 grams for the zinc gluconate. Still further, the amount of the zinc ions in 100 grams of the antimicrobial hand wash composition at a level of 0.05 percent is 2.40e−4 grams for the zinc chloride. In this example, the zinc chloride contributes the most zinc ions to the antimicrobial hand wash composition. However, adding zinc chloride is just as efficacious as not adding any zinc at all. As a result, adding zinc chloride in the antimicrobial hand wash composition may not be desirable. In some examples, the zinc salt forms between 0.01 and 1.00 weight percent of the antimicrobial hand wash composition.

The antimicrobial hand wash composition also includes moisturizer. As used in the present specification, the term moisturizer refers to moisturizers, humectants, emollients, or other components that increase the water-content level of the skin surface upon application. Moisturizers may be chemical agents which cause the skin to be softer. Moisturizers may act by reducing evaporation from the surface of the skin. Humectants may be used as moisturizers. Oils may be used as moisturizers. In some examples, the moisturizer forms between 1.00 and 3.00 weight percent of the antimicrobial hand wash composition.

The antimicrobial hand wash composition may also include a number of other components such as aesthetic ingredients. These components include a fragrance, a dye, an extract, or any other component that improves the customer experience. In some examples, the aesthetic ingredients forms between 0.00 and 2.00 weight percent of the antimicrobial hand wash composition.

The antimicrobial hand wash composition may also include a preservative. The preservative is used to prevent decomposition by microbial growth or undesirable chemical changes in the antimicrobial hand wash composition. In some examples, the preservative forms between 0.01 and 1.00 weight percent of the antimicrobial hand wash composition.

The antimicrobial hand wash composition may include an aqueous medium in which the cationic antimicrobial active ingredient, the number of surfactants, the hydrocolloid, the moisturizer, and the zinc salt are distributed. The aqueous medium may be water. In some examples, the aqueous medium forms between 87.7 and 97.87 weight percent of the antimicrobial hand wash composition. For example, the aqueous medium may form between 90 and 95 weight percent of the antimicrobial hand wash composition.

In some examples, the antimicrobial hand wash composition may be defined by the pH of the composition. In one example, the pH of the antimicrobial hand wash composition is between 5.00 and 6.40

In some examples, the antimicrobial hand wash composition is included in an antimicrobial hand wash product, in which the antimicrobial hand wash composition is included in a container. The container may be equipped with an opening capable of dispensing the antimicrobial hand wash composition. An opening in the container dispenses the antimicrobial hand wash composition by a plunger type mechanism, or another appropriate dispenser. The opening dispenses the antimicrobial hand wash composition onto an area of the skin. For example, the opening may dispense the antimicrobial hand wash composition onto a hand of a user, for application to the hands. Another type of dispensing container includes a simple opening allowing for the dispensing of the antimicrobial hand wash composition, which container may have a cap.

Table (1) gives an example of the antimicrobial hand wash composition as described in the present specification. More specifically, the antimicrobial hand wash composition of Table (1) includes the below listed ingredients in the indicated weight percentages.

TABLE (1)

| Ingredient | Weight Percent (%) |
|---|---|
| Water | 87.7-97.87 |
| Surfactant (Alkamine Oxide) | 1.00-2.00 |
| Co-Surfactant | 0.00-2.00 |
| Cationic Antimicrobial Active Ingredient | 0.10-0.30 |
| Hydrocolloid | 0.01-1.00 |
| Moisturizers | 1.00-3.00 |
| Zinc Salt | 0.01-2.00 |
| Fragrance, Dyes, Extracts, Additives | 0.00-2.00 |
| Preservative | 0.01-1.00 |

As illustrated in Table (1), the surfactant may be alkamine oxide. Further, the co-surfactant may be amphoteric or non-ionic.

Table (2) gives an example of the effectiveness of the antimicrobial hand wash composition for reducing bacteria, such as transient microbial flora, when using the health care personnel hand wash method and varying the type of zinc salt compound. Further, table (2) compares the antimicrobial hand wash of according to the present specification to a hand wash composition with no zinc and a hand wash composition with zinc chloride.

TABLE (2)

| | Log10 Reduction | Log10 Reduction |
|---|---|---|
| Compounds | After One Wash | After Ten Washes |
| No Zinc | 2.46 | 2.90 |
| Zinc Chloride | 2.53 | 2.90 |
| Zinc Gluconate | 2.59 | 3.10 |
| Zinc Sulfate | 2.61 | 3.40 |

As indicated in Table (2), the antimicrobial hand wash composition containing no zinc salts exhibits no more than a 2.90 log 10 reduction in bacteria on a skin surface after ten washes. Similarly, the antimicrobial hand wash composition containing zinc chloride exhibits no more than a 2.90 log 10 reduction in bacteria on a skin surface after ten washes. In contrast, the antimicrobial hand wash composition of the present specification containing zinc gluconate exhibits a 3.10 log 10 reduction in bacteria on a skin surface after ten washes. Similarly, the antimicrobial hand wash composition of the present specification containing zinc sulfate exhibits a 3.40 log 10 reduction in bacteria on a skin surface after ten washes. As a result, the antimicrobial hand wash composition is more effective when using zinc gluconate or zinc sulfate.

As demonstrated above, the antimicrobial hand wash composition is unique in that the amount of the different components, and more specifically the type of the zinc compound, lead to unexpectedly beneficial antimicrobial efficacy properties. Put another way, the antimicrobial hand wash composition is defined by the zinc salt properties, in addition to the above stated amounts of ingredients.

An antimicrobial hand wash composition that exhibits a 3 log 10 reduction in bacteria on a skin surface after ten hand washes may have increased customer appeal as a result of its ability to reduce bacteria on the skin surface after ten washes. Further, due to the moisturizers in the antimicrobial hand wash composition, the antimicrobial hand wash composition exhibits adequate lather and nice skin feel during and after use.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

1. An antimicrobial hand wash composition, comprising:
a cationic antimicrobial active ingredient;
a number of surfactants comprising at least two surfactants compatible with cationics with at least one of the surfactants comprising an alkamine oxide;
a hydrocolloid; and
a zinc salt comprising at least one of zinc sulfate and zinc gluconate,
wherein the composition has a pH ranging between 5.00 and 6.40.

2. The antimicrobial hand wash composition of claim 1, in which the antimicrobial hand wash exhibits a 3 log 10 reduction in bacteria on a skin surface after ten hand washes.

3. The antimicrobial hand wash composition of claim 1, in which the zinc salt has:
a log D value less than 0 at a pH of 5.5; and
a log P value less than 0.

4. The antimicrobial hand wash composition of claim 1, in which the antimicrobial hand wash composition has a molar ratio of zinc salt to a ligand that is between 3:20 and 7:20.

5. The antimicrobial hand wash composition of claim 1, further comprising moisturizers, a fragrance, a dye, an extract, a preservative, or combinations thereof.

6. The antimicrobial hand wash composition of claim 1, in which an amount of the zinc ions in 100 grams of the antimicrobial hand wash composition at a level of 0.05 percent is 7.17e−5 grams for the zinc gluconate.

7. The antimicrobial hand wash composition of claim 1, in which an amount of the zinc ions in 100 grams of the antimicrobial hand wash composition at a level of 0.05 percent is 1.14e−4 grams for the zinc sulfate.

8. An antimicrobial hand wash product, comprising:
a container; and
an antimicrobial hand wash composition disposed within the container, the antimicrobial hand wash comprising;
a cationic antimicrobial active ingredient;
a number of surfactants comprising at least two surfactants compatible with cationics with at least one of the surfactants comprising an alkamine oxide;
a hydrocolloid;
a moisturizer;
a zinc salt comprising at least one of zinc sulfate and zinc gluconate; and
an aqueous medium in which the cationic antimicrobial active ingredient, the number of surfactants, the hydrocolloid, the moisturizer, and the zinc salt are distributed,
wherein the composition has a pH ranging between 5.00 and 6.40.

9. The antimicrobial hand wash product of claim 8, in which the zinc salt has:
a log D value less than 0 at a pH of 5.5;
a log P value less than 0; and
the antimicrobial hand wash composition has a molar ratio of zinc salt to a ligand that is between 3:20 and 7:20.

10. The antimicrobial hand wash product of claim 8, in which an amount of the zinc ions in 100 grams of the antimicrobial hand wash composition at a level of 0.05 percent is 1.14e−4 grams for the zinc sulfate.

11. The antimicrobial hand wash product of claim 8, in which an amount of the zinc ions in 100 grams of the antimicrobial hand wash composition at a level of 0.05 percent is 7.17e−5 grams for zinc gluconate.

12. The antimicrobial hand wash product of claim 8, further comprising a fragrance, a dye, an extract, a preservative, or combinations thereof.

13. (canceled)

14. An antimicrobial hand wash composition, comprising:
a cationic antimicrobial active ingredient forming between 0.10 and 0.30 weight percent of the antimicrobial hand wash composition;
a number of surfactants comprising at least two surfactants compatible with cationics with at least one of the surfactants comprising an alkamine oxide forming between 1.00 and 2.00 weight percent of the antimicrobial hand wash composition;
a hydrocolloid forming between 0.01 and 1.00 weight percent of the antimicrobial hand wash composition;
a moisturizer forming between 1.00 and 3.00 weight percent of the antimicrobial hand wash composition; and
a zinc salt forming between 0.01 and 2.00 weight percent of the antimicrobial hand wash composition;
in which:
the zinc salt has a log D value less than 0 at a pH of 5.5;
the zinc salt has a log P value less than 0; and
the antimicrobial hand wash composition has a molar ratio of zinc salt to a ligand that is between 3:20 and 7:20, and
the antimicrobial hand wash composition has a pH ranging between 5.0 and 6.4.

15. The antimicrobial hand wash composition of claim 14, in which the zinc salt comprising at least one of zinc sulfate and zinc gluconate.

16. The antimicrobial hand wash composition of claim 15, in which an amount of zinc ions in 100 grams of the antimicrobial hand wash composition at a level of 0.05 percent is 1.14e−4 grams for the zinc sulfate.

17. The antimicrobial hand wash composition of claim 15, in which an amount of the zinc ions in 100 grams of the antimicrobial hand wash composition at a level of 0.05 percent is 7.17e−5 grams for the zinc gluconate.

18. The antimicrobial hand wash composition of claim 14, in which further comprising a fragrance, a dye, an extract, a preservative, or combinations thereof forming between 0.00 and 3.00 weight percent of the antimicrobial hand wash composition.

19. (canceled)

20. The antimicrobial hand wash composition of claim 14, in which the antimicrobial hand wash exhibits a 3 log 10 reduction in bacteria on a skin surface after ten hand washes
